(19) United States
(12) Patent Application Publication
(10) Pub. No.: US 2021/0101143 A1
(43) Pub. Date: Apr. 8, 2021

(54) CLINICAL SAMPLE STORAGE CASSETTES

(71) Applicant: Indian Institute of Science, Bangalore (IN)

(72) Inventor: Bhushan Jayant Toley, Bangalore (IN)

(21) Appl. No.: 15/733,088

(22) PCT Filed: Nov. 13, 2018
(86) PCT No.: PCT/IN2018/050747

§ 371 Date: May 13, 2020

(30) Foreign Application Priority Data
Nov. 13, 2017 (IN) 201741040468

(51) Int. Cl. B01L 3/00

(52) U.S. Cl. CPC B01L 3/5021; B01L 2400/0406; B01L 2300/0864; B01L 2300/0816

(57) ABSTRACT

A clinical sample storage cassette and an assembly comprising the clinical sample storage cassette is provided. The clinical sample storage cassette can comprise a top layer, a distributor layer, and a storage membrane. The top layer can comprise a first side to receive a clinical sample and a second side coupled with a distributor layer. The distributor layer is to receive the clinical sample from the top layer. The distributor layer can comprise a first distributor side coupled to the second side and a second distributor side coupled to the storage membrane to transfer the clinical sample to the storage membrane. The distributor layer and the storage membrane can have different flow rates of the clinical sample to allow for uniform flow and storage of the clinical sample on the storage membrane.

### TECHNICAL FIELD

The present subject matter relates in general to clinical sample storage and, in particular, to clinical sample storage cassettes.

### BACKGROUND

Clinical samples may sometimes need to be transported over long distances. Transportation time could last from days to weeks. In the absence of cold chains, samples may putrefy and become unfit for analysis. Typically, blood samples are used to test an individual for diseases. Generally, for long distance transportation of blood sample Dried Blood Spot (DBS) technology is used. DBS technology is a form of bio-sampling where blood samples are blotted and dried on a filter paper. The dried filter paper is then shipped for analysis, for example, by Deoxyribonucleic Acid (DNA) amplification, High Pressure Liquid Chromatography (HPLC), and the like. However, DBS technology has not been modified for collection, stabilization, and storage of other clinical samples, such as sputum, urine, and the like.

### BRIEF DESCRIPTION OF DRAWINGS

The detailed description is described with reference to the accompanying figures. In the figures, the left-most digit(s) of a reference number identifies the figure in which the reference number first appears. The same numbers are used throughout the drawings to reference like features and components.

FIG. 1 illustrates a schematic of a clinical sample storage cassette, in accordance with an implementation of the present subject matter.

FIG. 2(a) illustrates an example clinical sample storage cassette, in accordance with an implementation of the present subject matter.

FIG. 2(b) illustrates cross-section of the example clinical sample storage cassette, in accordance with an implementation of the present subject matter.

FIG. 3 depicts another example clinical sample storage cassette, in accordance with an implementation of the present subject matter.

FIG. 4 depicts yet another example clinical sample storage cassette, in accordance with an implementation of the present subject matter.

FIG. 5(a) depicts a front-view of an assembly for storing clinical samples, in accordance with an implementation of the present subject matter.

FIG. 5(b) depicts a side-view of the assembly for storing clinical samples, in accordance with an implementation of the present subject matter.

FIG. 6 depicts an illustration of a method for collecting, introducing and storing clinical samples, in accordance with an implementation of the present subject matter.

FIG. 7 depicts an illustration of method for analyzing clinical sample, in accordance with an implementation of the present subject matter.

FIG. 8(a) depicts comparison of distribution characteristic of a control device and a first cassette, in accordance with an implementation of the present subject matter.

FIG. 8(b) depicts comparison of distribution characteristic of a control device and a second cassette, in accordance with an implementation of the present subject matter.

FIG. 9 depicts results of the viscosity study on extent of rehydration of reagents, in accordance with an implementation of the present subject matter.

FIG. 10 depicts effect of material of fabrication on distribution and rehydration of reagents, in accordance with an implementation of the present subject matter.

### DETAILED DESCRIPTION

The present subject matter provides clinical sample storage cassettes for storage of clinical samples.

Different clinical samples, such as blood, sputum, urine, and the like, can be used for analysis and diagnosis of diseases and disorders. In certain conditions, such as if the clinical sample is collected from an individual in a resource deficient area, the same may be transported over long distances for analysis. Paper-based stabilizing technologies have been used to collect, stabilize, and transport clinical samples over long distances.

Currently existing paper-based stabilizing technologies, such as Dried Blood Spot (DBS) technology, can be used for collection of quantities of blood sample significantly less than 1 mL, typically 40-50 microliters. Generally, paper-based stabilizing technologies include a chemically treated paper for collection of the clinical sample. The chemically treated paper is generally impregnated with reagents, such as stabilizing agents, preservatives, antimicrobials and the like, and dried. When the chemically treated paper is contacted with the clinical sample, due to capillary forces, the clinical sample is wicked into pores present in the chemically treated paper. Generally, the reagents impregnated in the chemically treated paper are contained in the pores. The reagents in the pores dissolve and rehydrate as the clinical sample wicks through the chemically treated paper.

Typically, dissolution and rehydration of the reagents causes the reagents to be pushed along with the clinical sample away from a point of introduction of the clinical sample on the chemically treated paper. The reagents are pushed towards peripheries of the chemically treated paper. As the reagents are concentrated towards the peripheries, the clinical sample which lags behind during flow on the chemically treated paper is unable to interact with the reagents. This leads to unequal sample preparation on the chemically treated paper. Due to unequal sample preparation, the clinical sample may not be adequately stabilized for long-distance transportation and long-term storage.

On rehydration, the chemically treated paper is then dried prior to transportation, generally, by air drying. Therefore, there is a risk of exposure to the blood sample while the clinical sample dries. Additionally, for analysis, the chemically treated paper comprising the clinical sample thereon is punched, for example, by manually punching holes using a punching machine. Therefore, even during analysis, there is a risk of exposure to any pathogens present in the clinical sample.

The present subject matter provides clinical sample storage cassettes, assemblies comprising the clinical sample storage cassettes, and methods of collection and analysis of samples using the clinical sample storage cassettes. An example clinical sample storage cassette can comprise a top layer, a distributor layer, and a storage membrane. The top layer can receive a clinical sample on a first side and transfer the clinical sample to the distributor layer through a second side opposite to the first side. For this, the second side can be coupled with a first distributor side of the distributor layer.

The distributor layer can thus receive the clinical sample from the top layer on the first distributor side. A second distributor side, which is opposite to the first distributor side, can be coupled to the storage membrane to transfer the clinical sample to the storage membrane.

The storage membrane can receive the clinical sample from the distributor layer and store the clinical sample. The distributor layer and the storage membrane can be adapted so that the flow rate of the clinical sample through the distributor layer is greater than the flow rate of the clinical sample through the storage membrane to allow for uniform storage of the clinical sample on the storage membrane.

Various aspects of the present subject matter provide for different configurations of the distributor layer. In one example, the distributor layer can comprise a plurality of sub-distributor layers including a top-most sub-distributor layer, a bottom-most sub-distributor layer, and a plurality of intermediate layers disposed between the top-most distributor layer and the bottom-most distributor layer. In one example, each of the plurality of sub-distributor layers can comprise distributor ports. In another example, each of the plurality of sub-distributor layers may be porous membranes wherein the flow rate of the clinical sample through the plurality of sub-distributor layers increases from the top-most sub-distributor layer to the bottom-most sub-distributor layer.

In yet another example, the distributor layer can comprise a fluid channel. The fluid channel can comprise a main channel and a plurality of sub-channels. The main channel can receive the clinical sample from the top layer. The plurality of sub-channels extends away from the main channel and can receive the clinical sample from the main channel. Various other embodiments of the distributor layer will be evident to a person skilled in the art based on the teachings of the present application and are intended to be covered herein.

In an example, for ease of transportation and processing, the present subject matter also provides assemblies comprising the clinical sample storage cassette. An example assembly can comprise a container, such as a centrifuge tube, to hold the clinical sample storage cassette. The assembly can comprise a desiccant provided within the container to dry the storage membrane. In one example, by using the assembly, the clinical sample on the storage membrane can be directly processed without human contact. For example, the storage membrane can be treated with buffers within the centrifuge tube to extract the clinical sample from the storage membrane.

The clinical sample storage cassette of the present subject matter can be used for storage of clinical samples of quantities greater than 1 mL. Further, it can also be used for stabilization and storage of various clinical samples, such as sputum, urine, blood, and the like.

When the clinical sample is introduced at the top layer, due to capillary action, the clinical sample flows through the distributor layer to the storage membrane. Due to higher flow rate of the clinical sample in the distributor layer, the clinical sample spreads through the distributor layer and, thereby, gets uniformly transferred to the storage membrane. Thus, the higher flow rate of the clinical sample in the distributor later helps to distribute the clinical sample across the storage membrane This further helps in uniformly mixing the clinical sample with the reagents provided on the storage membrane which can help in improved stabilization and shelf-life of the clinical sample. Therefore, the clinical sample can be stored for an extended period of time without leading to degradation of the clinical sample. The clinical sample storage cassette, therefore, helps in eliminating the requirement of cold chains, reducing risk of exposure, and increasing volume of clinical sample that can be stored and transported.

The above and other features, aspects, and advantages of the subject matter will be better explained with regard to the following description and accompanying figures. It should be noted that the description and figures merely illustrate the principles of the present subject matter along with examples described herein and, should not be construed as a limitation to the present subject matter. It is thus understood that various arrangements may be devised that, although not explicitly described or shown herein, embody the principles of the present disclosure. Moreover, all statements herein reciting principles, aspects, and examples thereof, are intended to encompass equivalents thereof. Further, for the sake of simplicity, and without limitation, the same numbers are used throughout the drawings to reference like features and components.

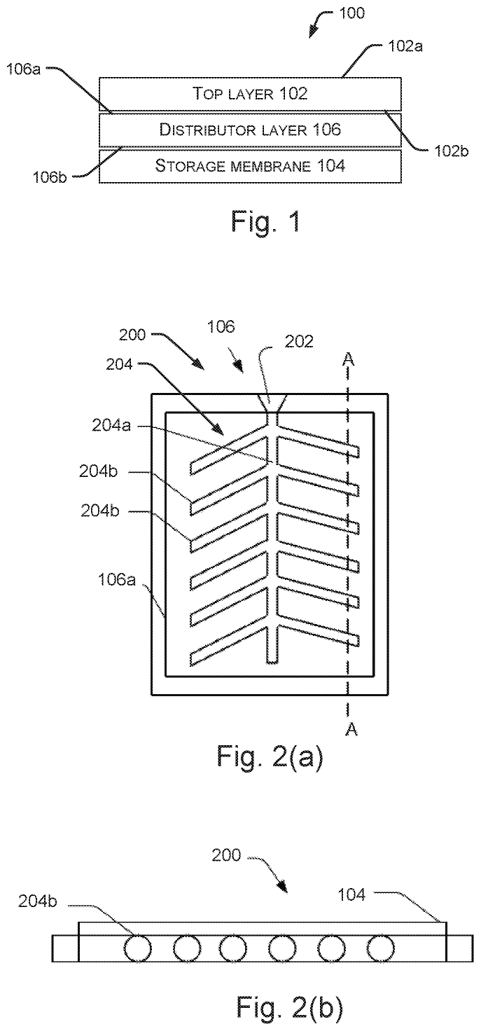

FIG. 1 illustrates an example clinical sample storage cassette 100, in accordance with an implementation of the present subject matter. The clinical sample storage cassette 100 can be used to store and transport any kind of clinical sample, for example, blood, urine, sputum, and the like. The clinical sample may be pre-processed prior to being stored and transported using the clinical sample storage cassette 100.

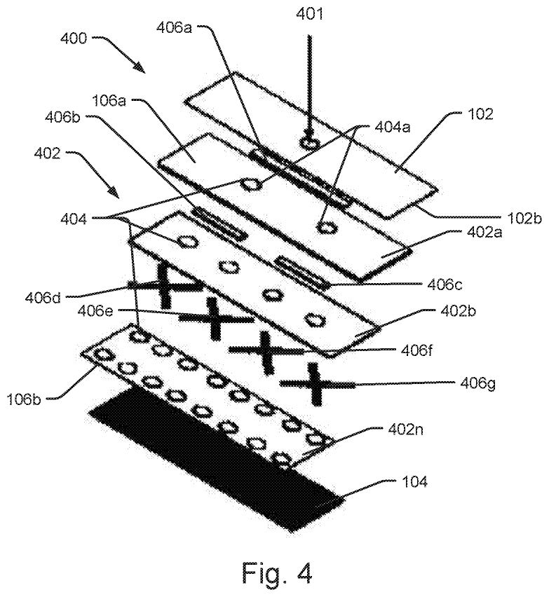

The clinical sample storage cassette 100 can comprise a top layer 102, a storage membrane 104 and a distributor layer 106 coupled to and disposed between the top layer 102 and the storage membrane 104. The top layer 102 can comprise a first side 102a and a second side 102b opposite to the first side 102a. The first side 102a can be used for receiving the clinical sample. The first side 102a can comprise a sample inlet port for introduction of the clinical sample into the clinical sample storage cassette. The sample inlet port is explained later with reference to FIG. 3 and FIG. 4. In an example, the top layer 102 can be fabricated from acrylic plastic.

The second side 102b of the top layer 102 can be coupled to the distributor layer 106. The distributor layer 106 can thus receive the clinical sample from the top layer 102. The distributor layer 106 can comprise a first distributor side 106a and a second distributor side 106b opposite to the first distributor side 106a. The first distributor side 106a can be coupled to the second side 102b of the top layer 102. The second distributor side 106b can be coupled to the storage membrane 104 to transfer the clinical sample from the top layer 102 to the storage membrane 104.

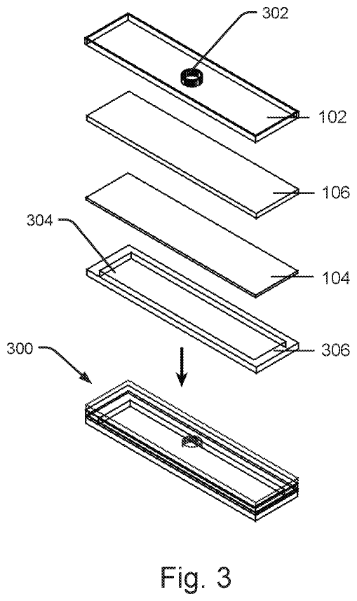

The distributor layer 106 can be used to distribute the clinical sample uniformly over the storage membrane 104, thereby reducing movement of the clinical sample through the storage membrane 104 and reducing the movement of reagents in the storage membrane. Various configurations of the distributor layer 106 are possible to achieve this. In one example, the distributor layer 106 can comprise a fluid channel. The fluid channel can include a main channel and a plurality of sub-channels extending away from the main channel The fluid channel and the plurality of sub-channels is explained later with reference to FIG. 2(a) and FIG. 2(b). In another example, the distributor layer 106 may be fabricated from a porous material, such as glass fiber, through which the clinical sample can be distributed by wicking/ capillary action. In yet another example, the distributor layer 106 may be fabricated from a non-porous material, such as acrylic plastic, and the clinical sample may be transferred from the distributor layer 106 to the storage membrane 104 via distributor ports provided on the distributor layer 106.

In yet another example, the distributor layer 106 can comprise a plurality of sub-distributor layers. The plurality of sub-distributor layers is explained later with reference to FIG. 3 and FIG. 4.

The distributor layer 106 can transfer clinical sample from the top layer 102 to the storage membrane 104. The storage membrane 104 can receive the clinical sample from the distributor layer 106 and to store the clinical sample. The storage membrane 104 can be fabricated from one of cellulose, nitrocellulose, glass fiber membrane and the like. The storage membrane 104 can be impregnated with reagents, such as DNA stabilizing agents, chaotropic agents, reducing agents, antimicrobial and antifungal agents, and combinations thereof to help store the clinical sample, for example, while it is transported from point of collection to point of analysis. In one example, to increase a volume of clinical sample that can be stored on the storage membrane 104, the stabilizing agent that may be used is trehalose. However, as will be understood, other stabilizing agents may also be used.

It will be understood that the material, porosity/size of ports, and other properties of the distributor layer 106 and the storage membrane 104 can be selected appropriately based on the viscosity and other characteristics of the clinical sample so as to obtain uniform distribution and storage of the clinical sample in the clinical sample storage cassette 100. For example, when the clinical sample is sputum, which is generally thick and viscous, greater pore size/port size may be used in the distributor layer 106 as compared to when the clinical sample is blood or urine which are less viscous.

The distributor layer 106 and the storage membrane 104 can allow for different flow rates of clinical sample. Specifically, the flow rate of clinical sample through the distributor layer 106 can be substantially greater than the flow rate of the clinical sample through the storage membrane 104. The difference in flow rate of the clinical sample in the distributor layer 106 and the storage membrane 104 allows for uniform distribution and storage of the clinical sample in the storage membrane 104. Due to the uniform distribution, reagents impregnated on the storage membrane 104 are not pushed to the periphery when the clinical sample is transferred from the distributor layer 106 to the storage membrane 104. This, therefore, helps in better mixing of the clinical sample with the reagents, enhancing the stability of the clinical sample stored therein. The use of the clinical sample storage cassette 100 also reduces risk of cross-contamination and exposure during collection and analysis as will be explained later.

In an example, the top layer 102, the distributor layer 106, and the storage membrane 104 may be coupled using a Pressure Sensitive Adhesive (PSA) tape between the top layer 102, the distributor layer 106, and the storage membrane 104. However, it is to be understood that other mechanisms may be used to couple the top layer 102, the distributor layer 106, and the storage membrane 104.

In an example, the clinical sample storage cassette 100 can comprise a supporting layer (not shown) coupled to the storage membrane 104 on a side opposite to the distributor layer 106. The supporting layer may be fabricated from acrylic plastic. The supporting layer can help provide mechanical support to the clinical sample storage cassette 100 when the clinical sample is introduced on the first side 102a of the top layer 102.

Examples of the clinical sample storage cassette are explained with reference to FIG. 2(a), FIG. 2(b), FIG. 3, and FIG. 4.

FIG. 2(a) illustrates an example distributor layer 106 of an example clinical sample storage cassette 200, in accordance with an implementation of the present subject matter. FIG. 2(b) illustrates cross-section of the example clinical sample storage cassette 200, in accordance with an implementation of the present subject matter.

FIG. 2(a) depicts the distributor layer 106 of the clinical sample storage cassette 200. The distributor layer 106 can comprise a distributor port 202 to receive the clinical sample form the top layer 102 (not shown). As mentioned previously, the distributor layer 106 may be fabricated from glass fibers or non-porous material, such as acrylic plastic. As shown in FIG. 2(a), the distributor layer 106 can comprise a fluid channel 204. The fluid channel 204 can be formed on the distributor layer 106, for example, by techniques such as, printing, soft-lithography, photolithography, deposition, CNC based micro-milling, and the like.

The fluid channel 204 can comprise a main channel 204a and a plurality of sub-channels 204b. The main channel 204a can extend substantially through a mid-portion of the distributor layer 106. The plurality of sub-channels 204b branch out extend away from the main channel 204a. Although FIG. 2(a) depicts only a single set of sub-channels 204b extending from the main-channel 204a, it is to be understood that the plurality of sub-channels 204b can further comprise sub-sub-channels branching out therefrom. Further, the pattern of the plurality of sub-channels 204b as shown in FIG. 2(a) should be not be construed as limiting. The pattern can be suitably modified as will be understood.

The main channel 204a can be coupled to the distributor port 202 which can be coupled to a sample introduction/inlet port in the top layer 102. Although not shown in FIG. 2(a), it will be understood that the top layer 102 can be coupled to the distributor layer 106. The distributor port 202 can receive the clinical sample from the sample introduction port in top layer 102. The plurality of sub-channels 204b can receive the clinical sample from the main channel 204a. In an example, the clinical sample can be homogenized prior to introduction in the clinical sample storage cassette 200. On introduction of the clinical sample, due to capillary action, the clinical sample flows through the main channel 204a and the plurality of sub-channels 204b, thus getting distributed across the distributor layer 106.

The distributor layer 106 can be coupled to the storage membrane 104 as shown in FIG. 2(b). FIG. 2(b) depicts cross-sectional view of the clinical sample storage cassette 200 along line A-A of FIG. 2(a). It is to be understood that the cross section of the plurality of sub-channels 204b shown in FIG. 2(b) may be circular or rectangular. In an example, the storage membrane 104 can extend throughout a length of the distributor layer 106. In another example, the storage membrane 104 may extend only for a portion of the distributor layer 106. The storage membrane 104 can be cut to the required size, for example, by using a CO₂ laser cutting. However, it is to be understood that other techniques may be used as well.

The storage membrane 104 can be fabricated from one of cellulose, nitrocellulose, glass fiber membrane and the like. The storage membrane 104 can be impregnated with reagents, such as DNA stabilizing agents, chaotropic agents, reducing agents, antimicrobial and antifungal agents, and combinations thereof.

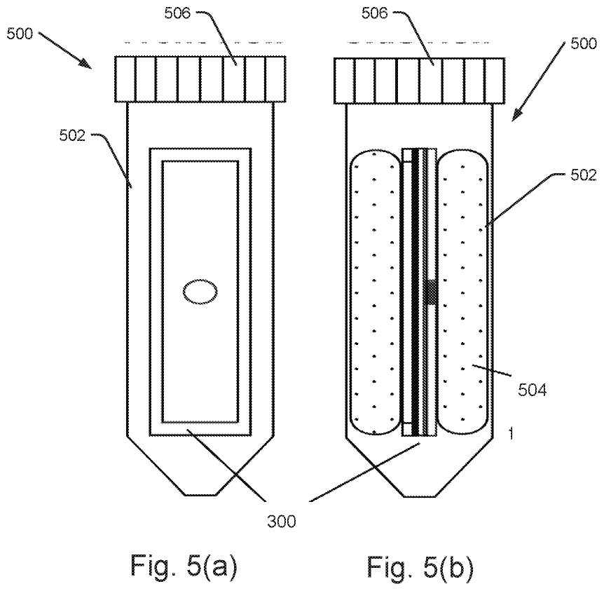

In operation, clinical sample received at the distributor port 202 is received by the main channel 204a. Due to capillary forces, the clinical sample flows through the main channel 204a and through the plurality of sub-channels 204b. As the storage membrane 104 is in contact with the distributor layer 106, the clinical sample wicks through the storage membrane 104 and is stored thereon. As the flow rate of the clinical sample through the distributor layer 106 is greater than through the storage membrane 104, the clinical sample spreads faster through the distributor layer 106 and therefore, the wicking of the clinical sample into the storage membrane 104 happens substantially uniformly across the storage membrane 104. Reagents in the storage membrane 104 stabilize the clinical sample. Thus, the clinical sample storage cassette 200 as shown in FIGS. 2(a) and 2(b) helps in ensuring that the clinical sample is uniformly distributed over the storage membrane 104 without altering the spatial distribution and concentration of the reagents impregnated on the storage membrane 104. While the clinical sample storage cassette 200 can be used as an individual unit, the clinical sample storage cassette 200 can also be part of an assembly as shown in FIGS. 5(a) and 5(b).

FIG. 3 depicts another example clinical sample storage cassette 300, in accordance with an implementation of the present subject matter. As shown in FIG. 3, the clinical sample storage cassette 300 can comprise the top layer 102, storage membrane 104, and the distributor layer 106 disposed between the top layer 102 and the storage membrane 104.

The top layer 102 can comprise a sample inlet port 302. The clinical sample can be introduced into the clinical sample storage cassette 300 at the sample inlet port 302. The clinical sample may be introduced, for example, by injecting or by micro-pipetting. The clinical sample introduced can be received by the distributor layer 106 from the top layer 102. The clinical sample flows through and spreads across the distributor layer 106. In one example, the distributor layer 106 is fabricated from a porous material, such as glass fiber. Due to the presence of pores, the clinical sample can be transferred from the first distributor side 106a to the second distributor side 106b and wicked by the storage membrane 104.

As discussed above, the distributor layer 106 can have a flow rate of clinical sample much higher than a flow rate of the clinical sample in the storage membrane 104. Due to different flow rates of the clinical sample in the distributor layer 106 and the storage membrane 104, the clinical sample can be uniformly stored in the storage membrane 104. As explained previously, this ensures uniform mixing of the clinical sample with the reagents provided on the storage membrane 104 and enhanced stability of the clinical sample during transport.

To provide mechanical stability to the clinical sample storage cassette 300, a supporting layer 304 can be provided opposite coupled to the storage membrane 104 at a side opposite to the distributor layer 106. The supporting layer 304 can be fabricated from acrylic plastic. The supporting layer 304 can comprise a rim 306 provided along its sides to hold the storage membrane 104 and the distributor layer 106. In an example, pores can be provided in the supporting layer 304, for example, at the rim 306 to allow for drying of the storage membrane 104.

In operation, clinical sample is received at the sample inlet port 302 of the top layer 102. Due to capillary forces, the clinical sample flows along the first distributor side 106a and due to permeability of the distributor layer 106, the clinical sample is flows through the distributor layer 106 to the second distributor side 106b. On the second distributor side 106b, the clinical sample can be wicked into and stored in the storage membrane 104. Due to difference in flow rate of the clinical sample in the distributor layer 106 and the storage membrane 104, the clinical sample is distributed uniformly across the storage membrane 104.

FIG. 3 depicts the clinical sample storage cassette 300 comprising a single distributor layer 106. However, the distributor layer 106 can comprise a plurality of sub-distributor layers as explained with reference to FIG. 4.

FIG. 4 depicts yet another example clinical sample storage cassette 400, in accordance with an implementation of the present subject matter. The clinical sample storage cassette 400 comprises the top layer 102 comprising the sample inlet port 401 to receive the clinical sample.

As shown in FIG. 4, the distributor layer 106 (not shown) can comprise a plurality of sub-distributor layers 402. The plurality of sub-distributor layers 402 can comprise a top-most sub-distributor layer 402a and a bottom-most sub-distributor layer 402n. The top-most sub-distributor layer 402a can have the first distributor side 106a coupled to the second side 102b of the top layer 102. The bottom-most sub-distributor layer 402n can have the second distributor side 106b coupled to the storage membrane 104.

The plurality of sub-distributor layers 402 can also comprise a plurality of intermediate layers disposed between the top-most sub-distributor layer 402a and the bottom-most sub-distributor layer 402n. FIG. 4 depicts a single intermediate layer 402b between the top-most sub-distributor layer 402a and the bottom-most sub-distributor layer 402n. However, it is to be understood that any number of intermediate layers may be provided based on the clinical sample used, quantity of clinical samples that is to be stored on the storage membrane 104, and the like.

In one example, each sub-distributor layer of the plurality of sub-distributor layers 402 is formed from a porous material fabricated from glass fibers. In another example, each sub-distributor layer is fabricated from a material such that flow rate of the clinical sample in a preceding sub-distributor layer is greater than in a subsequent sub-distributor layer that receives the clinical sample from the preceding sub-distributor layer. For example, the top-most sub-distributor layer 402a may be fabricated from material having a higher flow rate than the intermediate sub-distributor layer 402b and so on.

In an example, each sub-distributor layer of the plurality of sub-distributor layer 402 can be fabricated from a non-porous material, such as acrylic plastic. In said example, each sub-distributor layer of the plurality of sub-distributor layers 402 can comprise distributor ports 404 to transfer clinical sample to subsequent sub-distributor layers. The distributor ports 404 of each sub-distributor layer can receive clinical sample from a preceding layer. For example, top-most sub-distributor layer 402a can receive the clinical sample from the top layer 102, the intermediate layer 402b can receive the clinical sample from the top-most sub-distributor layer 402a and so on.

In an example, a number of distributor ports 404 in the top-most sub-distributor layer 402a, the plurality of intermediate layers, and the bottom-most sub-distributor layer 402n increases exponentially by an even power of 2. For example, with reference to FIG. 4, the top-most sub-distributor layer 402a can have 2 distribution ports, the intermediate layer 402b can have 4 distribution ports, and the bottom-most sub-distributor layer 402n can have 16 distribution ports. However, depending on a number of intermediate layers, the number of distribution ports on each sub-distributor layer of the plurality of distributor layer 402 may vary, as will be understood.

In one example, the top layer 102, the distributor layer 106, and the storage membrane 104 can be coupled by using PSA tape. The PSA tape may be provided between the layers such that the tape does not block the distribution ports.

For example, as shown in FIG. 4, the top-most sub-distributor layer 402a can be coupled to the top layer 102 by using a single strip 406a of the PSA tape between the two distributor ports of the top-most sub-distributor layer 402a without blocking the distributor ports 404a. End of the single strip 406a can be provided such that they are close to the distributor ports 404a but do not block the distributor ports 404a.

Similarly, two strips 406b and 406c can be used to couple the intermediate sub-distributor layer 402b to the top-most sub-distributor layer 402a. Further, the bottom-most sub-distributor layer 402n can be coupled to the intermediate layer 402b by using four X-shaped tape pieces 406d, 406e, 406f, and 406g. Each edge of each X-shaped tape piece can correspond to a distribution port of the bottom-most sub-distributor layer 402n. It is to be understood that other configurations and shapes of the PSA tape may be used to couple the different layers, as will be understood. Similar to the clinical sample storage cassette 300, a supporting layer may also be provided coupled to the storage membrane 104.

In operation, clinical sample received at the sample inlet port 401 of the top layer 102. Due to capillary forces, the clinical sample flows along the first distributor side 106a of the top-most sub-distributor layer 402a and subsequent sub-distributor layers, namely, the intermediate sub-distributor layer 402b and the bottom-most sub-distributor layer 402n. In an example, the clinical sample flows through subsequent sub-distributor layers through the distributor ports 404 in the sub-distributor layers. On the second distributor side 106b of the bottom-most sub-distributor layer 402n, the clinical sample can be wicked into and stored in the storage membrane 104. Due to difference in flow rate of the clinical sample in each of the plurality of sub-distributor layer 403 and the storage membrane 104 and the distributor ports in the bottom-most sub-distributor layer 402n, the clinical sample is distributed uniformly at different spots across the storage membrane 104. The spots can correspond to the distribution ports of the bottom-most sub-distributor layer 402n.

In one example, to further reduce chances of contamination, the clinical sample storage cassette 100, 200, 300, and 400 can be provided as part of an assembly. FIG. 5(a) depicts a front-view of an assembly 500 for storing clinical samples, in accordance with an implementation of the present subject matter. FIG. 5(b) depicts a side-view of the assembly 500 for storing clinical samples, in accordance with an implementation of the present subject matter.

The assembly 500 can comprise a container 502 to hold the clinical sample storage cassette. For the sake of brevity, assembly 500 is explained with reference to clinical sample storage cassette 300. However, it is to be understood that various configurations of clinical sample storage cassette 100, such as 200, 400, or other similar configurations, may also be used.

In the assembly 500, the clinical sample storage cassette 300 can be held within the container 502. In an example, the container 502 is a centrifuge tube. Therefore, the clinical sample storage cassette 300 can be fabricated so as to fit into the container 502. In an example, the container 502 can be provided with a desiccant pack 504. The desiccant pack 504 can be provided within the container 502 to dry the storage membrane 104, thereby, allowing storage of the clinical sample for an extended period of time. In an example, the desiccant used in the desiccant pack 504 may be silica gel, however in other examples, activated alumina, bentonite clay, calcium chloride, calcium sulfate, or molecular sieves may be used. The desiccant may also be chosen such that it changes color based on its moisture content.

To ensure negligible risk of exposure, the container 502 can be provided with a lid 506. The lid 506 can be, for example, screwed onto or press-fitted on the container 502 once the clinical sample has been collected and introduced into the container 502.

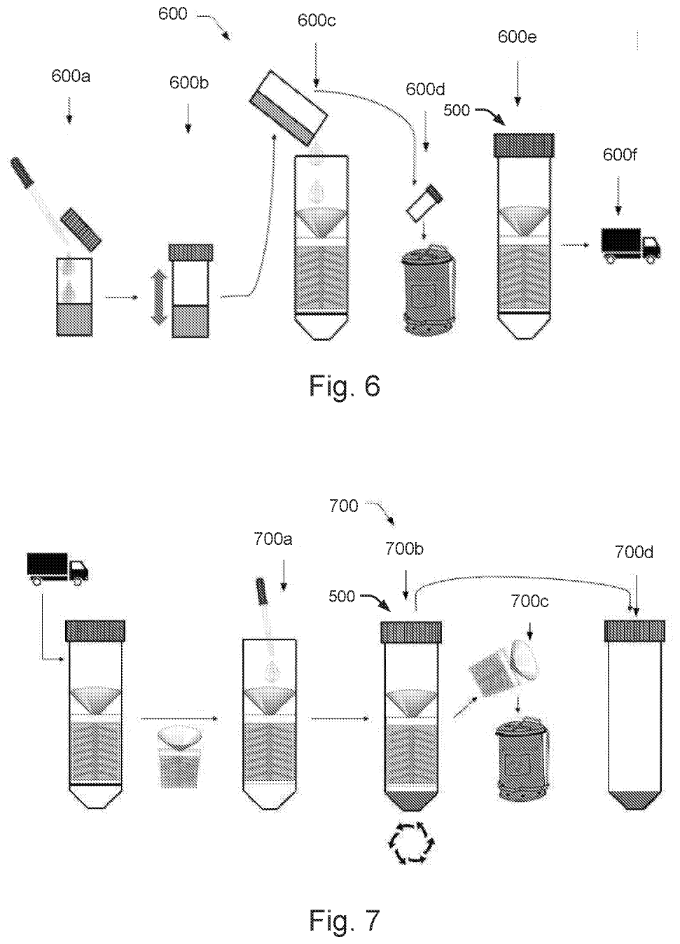

The present subject matter also provides a method for collection and analysis of clinical sample. The method is explained with reference to FIG. 6 and FIG. 7. FIG. 6 depicts an illustration of a method 600 for collecting, introducing and storing clinical sample in the assembly 500, in accordance with an implementation of the present subject matter. The method 600 has been depicted with respect to sputum as the clinical sample. However, other clinical samples, such as blood, urine, saliva may also be used. Further, in the assembly 500 as shown in FIGS. 6 and 7, the clinical sample storage cassette 200 is shown. However, it is to be understood that clinical sample storage cassette 100, 300 and 400 may also be used.

As depicted in step 600a, the method comprises introducing collected sputum sample into a homogenization tube containing a homogenization solution. In an example, the homogenization solution comprises dithiothreitol (DTT). At step 600b, mixture of the sputum sample and the homogenization solution are mixed. In an example, the mixing is done manually. However, other methods of mixing known in the art may be used as well. The mixture of sputum sample and the homogenization solution can be allowed to rest for a period of about 10 mins.

At step 600c, the mixture comprising the clinical sample can be received by the clinical sample storage cassette 200. In an example, a funnel may be used to transfer the mixture to the clinical sample storage cassette 200. However, micropipette, dropper, and the like may also be used as well as will be understood. At step 600d, the homogenization tube is decontaminated and suitably discarded. At step 600e, the lid 506 of the container 502 containing the clinical sample storage cassette 200 can be closed. The storage membrane 104 comprising the clinical sample can be dried by the desiccant pack 504 provided within the container 502. At 600f, the container 502 can be transported. In an example, the container 502 may be transported to an analytical laboratory for further analysis, for example, for diagnosis of diseases.

FIG. 7 depicts an illustration of method 700 for analyzing clinical sample contained in the assembly 500, in accordance with an implementation of the present subject matter. At step 700a, an elution buffer can be received by the container 502. In an example, the elution buffer is one of Phosphate Buffer Saline (PBS), tris buffer, tris ethylenediaminetetraacetic acid (tris EDTA) buffer, triethanolamine buffer or other buffers compatible with polymerase chain reaction (PCR) analysis of DNA.

At step 700b, the container 502 can be centrifuged to extract the clinical sample from the storage membrane 104 to obtain a remnant fluid. The remnant fluid can comprise a mixture of the elution buffer and the clinical sample. At step 700c, the clinical sample storage cassette 200 can be discarded and at step 700d, remnants of centrifugation can be provided for further analysis, for example, for GeneXpert MTB/RIF testing.

Therefore, the clinical sample storage cassette 100, 200, 300, 400, can be used for storage of clinical samples of quantities greater than 1 mL. It can also be used for stabilization and storage of sputum, urine, blood, saliva and the like for an extended period of time by preventing putrefaction of the sample. Further, drying of potentially infectious sample in air and punching of paper to obtain discs is eliminated. This, thereby, reduces chances of spread of infections and contamination.

The present subject matter will now be illustrated with working examples, which are intended to illustrate the working of disclosure and not intended to be taken restrictively to imply any limitations on the scope of the present disclosure. Unless defined otherwise, all technical and scientific terms used herein have the same meaning as commonly understood to one of ordinary skill in the art to which this disclosure belongs. It is to be understood that this disclosure is not limited to the particular methods and experimental conditions described, as such methods and conditions may vary depending on the process and inputs used as will be easily understood by a person skilled in the art.

### EXAMPLES

### Example 1

### Distribution Characteristics Comparison

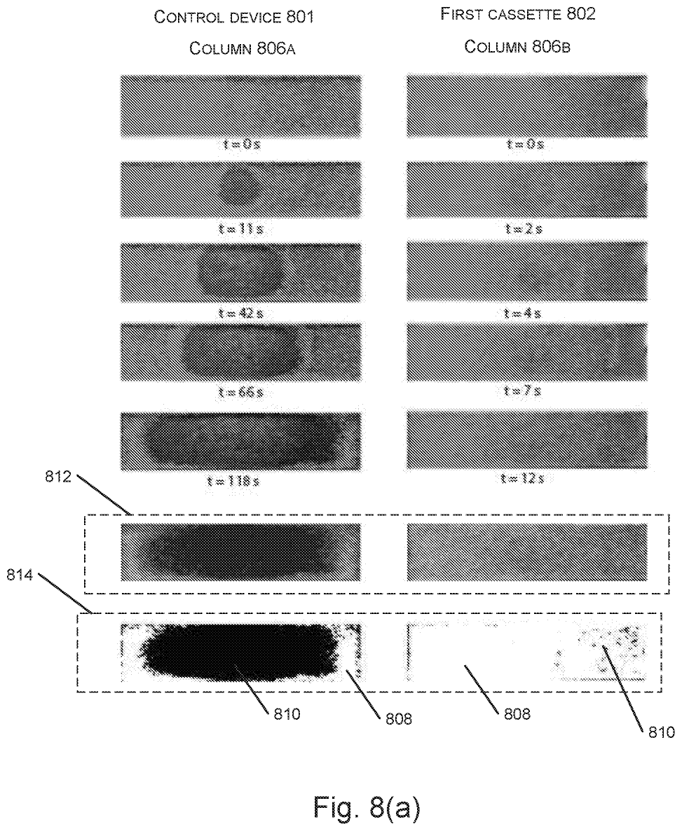

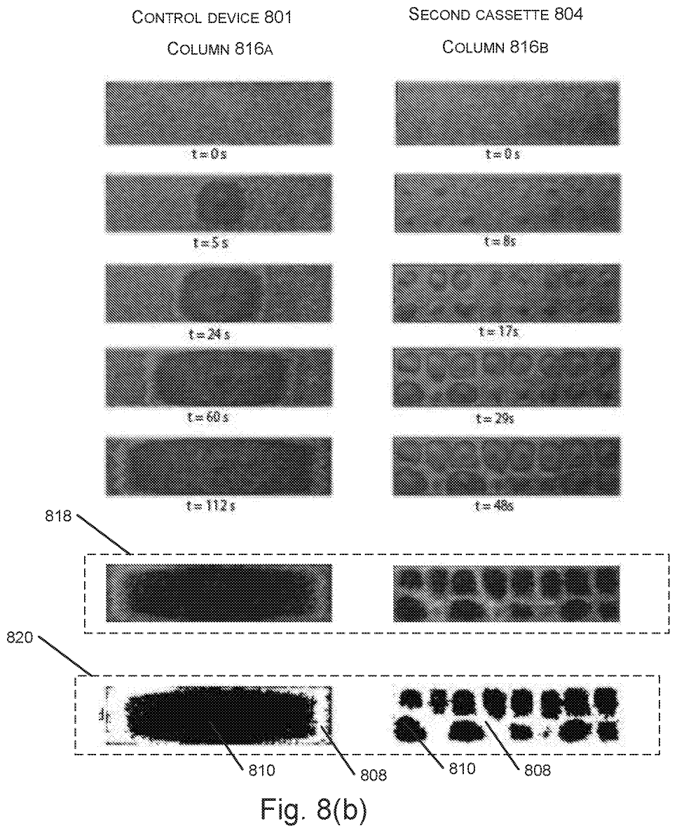

In this example, distribution characteristic of clinical sample between on a control device and the clinical sample storage cassette was compared. This example is explained with reference to FIGS. 8(a) and 8(b). The control device 801 in this example comprised the top layer 102 coupled directly to the storage membrane 104 at the first side 102a of the top layer 102. Two clinical sample storage cassettes were used in the study. The first cassette 802 comprised the single distributor layer 106 as depicted in FIG. 3 and the second cassette 804 comprises the plurality of distributor layers 106 as depicted in FIG. 4.

For studying distribution characteristics, the storage membrane in each of the control device 801, the first cassette 802, and the second cassette 804 was impregnated with a dye. The dye was representative of the reagents. The storage membrane used in each of the control device 801, the first cassette 802, and the second cassette 804 were Standard 17 membranes. The distributor layer(s) used in the first cassette 802 and the second cassette 804 were glass fibers.

### Example 1.1

### Distribution Characteristic Comparison Between Control Device and First Cassette

Experimental set-up for the study consisted of a rectangular slot (made of acrylic sheet) such that it can hold the control device 801 and the first cassette 802 under a common frame. It was also made sure that the experimental rectangular slot was at an elevation of 15 cm from the surface of the imaging station. To obtain images, a camera (Logitech webcam 10.0) was kept under the rectangular slot and it was adjusted in such a way that the control device 801 and the first cassette 802 can be captured under the same frame.

Fluid, for example, deionized water was introduced through sample inlet port in the control device 801 and the first cassette 802. In the control device 801, the fluid was wicked into the storage membrane 104. In the first cassette 802, due to capillary, the fluid flowed from the distributor layer to the storage membrane.

The color intensity of on the storage membranes of the control device 801 and the first cassette 802 was analyzed using ImageJ (NIH, USA). FIG. 8(a) depicts image of distribution of the fluid in the storage membrane of each of the control device 801 (indicated by column 806a) and the first cassette 802 (indicated by column 806b).

With reference to column 806a and 806b, at t=0 seconds, the fluid was injected through the sample inlet port of the top layer of the control device 801 and the first cassette 802. With reference to column 806a, the fluid rehydrates a large region near the central zone of the control device 801 at t=5 seconds and the extent of rehydration even increases further at t=24 seconds. As the time reaches 60 seconds, the fluid front reaches almost 50% of the distance in each half of the control device 801. In the control device 801, the fluid front exhibited the zone till which the rehydration has taken place. At t=112 seconds, the fluid front reaches almost near the edges of the control device 801 and hence, a large patch 810 is observed near the middle, depicting the maximum extent of rehydration. It is also observed that the sample-reagent interaction is concentrated mostly near the boundary or edges, indicated by 808, of the control device 801 at t=112 seconds.

With reference to column 806b, the various time frames of rehydration in the storage membrane of the first cassette 802 was studied. In the first cassette 802, as the distributor layer and the storage membrane had identical surface area and were in direct contact with each other, the rehydration occurred at the same rate along the storage membrane. This phenomenon occurred throughout the entire surface of the storage membrane.

This further results in the uniform interaction of the sample with the reagents on the surface of the storage membrane. The large patch 808 depicts the rehydration region on the storage membrane. In the storage membrane of the first cassette 802, it was observed that the fluid rehydrates almost 90% of the surface of the storage membrane in less than 7 seconds after the fluid is introduced in the first cassette 802. At time t=12 seconds maximum rehydration was seen in the first cassette 802. Overall, the time of rehydration was found to be significantly reduced and the rehydration was found to be highly uniform in contrast with the control device 801. The quantitative analysis of the uniformity in rehydration can be further described based on the mean and threshold studies.

Mean analysis with respect to the extent of rehydration of the first cassette 802 and the control device 801 was observed as demonstrated in row 812. The mean of the image of the stabilized zone of the first cassette 802 displayed an increase (mean=101.598) compared to the control device 801 (mean=31.495). Hence, there was an increase of almost 69% in the mean intensity of the first cassette 802 compared to the control device 801 (p<0.05).

The greater mean intensity in the first cassette 802 may be attributed to the enhanced interaction between the fluid and the reagents on the storage membrane. The threshold images, indicated by row 814, showed an improved visualization of the stabilization regime. The stable region occupied nearly 97.25% of the total area of the storage membrane in the first cassette 802. This was significantly higher than control device 801 where, almost 26.67% of the total area on the storage membrane corresponds to the stabilization zone. Therefore, there is an increment of nearly 72.5% of the stabilization area in the first cassette 802 compared to the control device 801 (p<0.05). Thus, based on the mean and threshold results, the first cassette 802 was observed to be more efficient and provided better stabilization chemistry compared to control device 801.

### Example 1.2

### Distribution Characteristic Comparison Between Control Device and Second Cassette

Experimental set-up for the study consisted of a rectangular slot (made of acrylic sheet) such that it can hold the control device 801 and the second cassette 804 under a common frame. It was also made sure that the experimental rectangular slot was at an elevation of 15 cm from the surface of the imaging station. To obtain images, a camera (Logitech webcam 10.0) was kept under the rectangular slot and it was adjusted in such a way that the control device 801 and the second cassette 804 can be captured under the same frame.

Fluid, for example, deionized water was introduced through sample inlet port in the control device 801 and the second cassette 804. In the control device 801, the fluid was wicked into the storage membrane 104. In the second cassette 804, due to capillary, the fluid flowed from the distributor layers to the storage membranes.

The color intensity of on the storage membranes of the control device 801 and the second cassette 804 was analyzed using ImageJ (NIH, USA). FIG. 8(b) depicts image of distribution of the fluid in the storage membrane of each of the control device 801 (indicated by column 816a) and the second cassette 804 (indicated by column 816b).

With reference to column 816a and 816b, at t=0 seconds, the fluid was injected through the sample inlet port of the top layer of the control device 801 and the second cassette 804. With reference to column 816a, the fluid rehydrates a large region near the central zone of the control device 801 at t=5 seconds and the extent of rehydration even increases further at t=24 seconds. As the time reaches 60 seconds, the fluid front reaches almost 50% of the distance in each half of the control device 801. In the control device 801, the fluid front exhibited the zone till which the rehydration has taken place. At t=118 seconds, the fluid front reaches almost near the edges of the control device 801 and hence, a large patch 808 is observed near the middle, depicting the maximum extent of rehydration. It is also observed that the sample-reagent interaction is concentrated mostly near the boundary or edges, indicated by 810, of the control device 801 at t=118 seconds.

With reference to column 816b, at t=8 seconds, rehydration begins to occur as fluid comes out from 16 different points on the storage membrane. As ‘t’ reaches 17 seconds, the rehydration zone grew in size. At t=29 seconds, almost 62.5% area of the storage membrane was rehydrated and a total rehydration of the second cassette 804 was found to occur at t=48 seconds. Hence, it was concluded that the total rehydration time was significantly less in the second cassette 804 compared to the control device 801. In addition, it was found that the sample-reagent interaction was enhanced for the second cassette 804 as the dye extended over a large domain on the storage membrane of the second cassette 804 in contrast to the control device 801.

Mean analysis with respect to the extent of rehydration in both the control device 801 and second cassette 804 was studied as indicated by row 818. The end point image of the control device 801 (t=112 s) and second cassette (t=48 s) was analyzed. The grey color region in the image depicts the stabilization zone which corresponds to the effective sample-reagent interaction. In contrast, the black colored zone represents the unstable region where the sample-reagent interaction is minimum. The mean intensity of the grey zone was found to increase from 24.433 to 48.488 (p<0.05) for the second cassette 804 as compared to the control device 801 with reference to row 818

Further, in order to effectively visualize the area of stabilization, the image was also thresholded as shown in row 820. The white region in the row 820 displays the area covered by the stabilization zone which is significantly higher for the second cassette 804 (area=48.55%) compared to the control device 801 (area=24.1%). Thus, it was observed that there was an enhancement of nearly 50.3% area of stabilization (p<0.05) for the second cassette 804 in contrast to the control device 801.

Overall, both the mean and threshold analysis clearly demonstrated that the stabilization zone enhanced significantly in the second cassette 804 in contrast to the control device 801.

### Example 2

### Effect of Viscosity

In this example, effect of viscosity of the fluid on extent of rehydration and stabilization was studied. Mock sputum was prepared by mixing 2% (w/v) of methylcellulose (Sigma M-0262) in 1000 mL of deionized water which resulted in a viscosity of 0.4 pa−s at 25° C. This viscosity matched that of naturally occurring mucus. The mock sputum was introduced into the control device 801, first cassette 802, and second cassette 804.

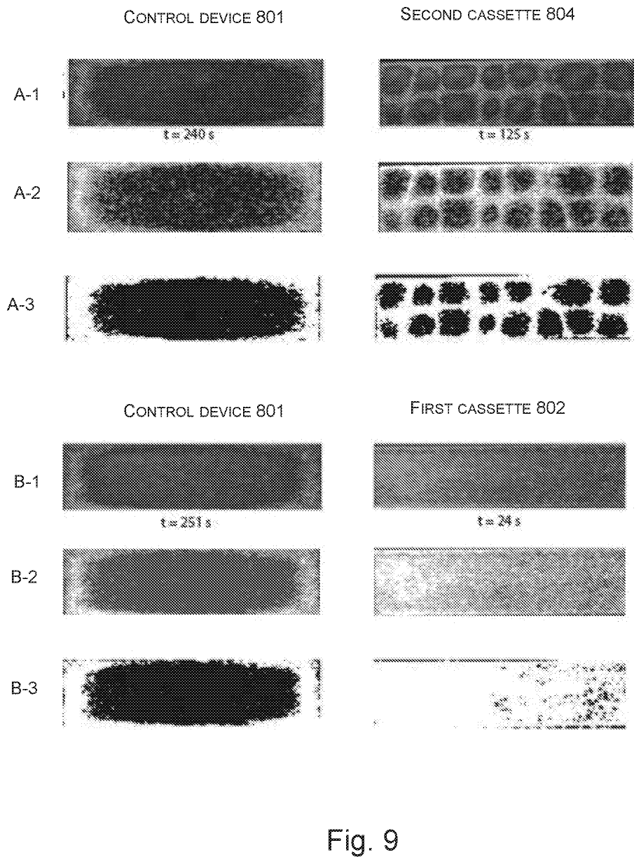

FIG. 9 depicts results of the viscosity study, in accordance with an implementation of the present subject matter. FIG. 9(A)-1 depicts end point time frame for rehydration of the dye using mock sputum on the storage membrane in the control device 801 and the second cassette 804; FIG. 9(A)-2 depicts mean intensity of the grey zone after rehydration in the control device 801 and the second cassette 804; FIG. 9(A)-3 depicts threshold studies after rehydration in the control device 801 and the second cassette 804.

FIG. 9(B)-1 depicts end point time frame for rehydration of the dye using mock sputum (viscosity=4 cP) on the storage membrane in the control device 801 and the first cassette 802. FIG. 9(B)-2 depicts mean intensity of the grey zone after rehydration in the control device 801 and the first cassette 802. FIG. 9(B)-3 depicts threshold studies after rehydration in the control device 801 and the first cassette 802.

As was expected, time taken to reach the maximum rehydration increased significantly for both the control device 801 (t=240s) and second cassette 804 (t=125s) in contrast to the previous case where the injected sample was water (control: t=112 s; second cassette 804: t=48 s) as seen in FIG. 9(A)-1. However, a slight increase in time for maximum rehydration was found to occur for the first cassette 802 (t=24 s) in contrast to the when deionized water was used (t=12 s).

Although the time taken for rehydration was higher, the images clearly demonstrated that viscosity of the clinical sample up to 4 cP, did not affect the rehydration or stabilization pattern in either of the first cassette 802 or the second cassette 804 compared to the previous cases where water (viscosity is 1 cP) was the injected sample. In general, it was concluded that the first cassette 802 was better compared to the second cassette 804, as the former displayed better rehydration and higher stabilization.

FIGS. 9(A)-2, 9(A)-3 and FIGS. 9(B)-2, 9(B)-3 demonstrated the mean and threshold results, respectively. FIG. 9(A)-2 illustrates that the mean intensity of the grey zone (stable region) in the control device 801 (mean=61.01) was significantly lesser and that can be attributed to the outward horizontal shifting and rehydration of the dye by the viscous sample from the central zone of the control device 801. In contrast, the second cassette 804 displayed greater mean intensity of grey zone (mean=98.08) as the rehydration occurred vertically through 16 different points at the same time and hence, the stabilization occurred almost throughout the entire surface of the storage membrane.

The threshold analysis displayed an increase in stabilization area by nearly 41.19% in the second cassette 804 compared to the corresponding the control device 801 (stabilization area=30.3%). The mean and threshold analysis of the first cassette 802 (mean=159.677, stabilization area=95%) demonstrated a significant increase by nearly 50.5% and 69.41%, respectively in contrast to the corresponding control device 801 (mean=79.029, stabilization area=29.06%). This was explained based on the direct contact and identical surface area of the distributor layer and storage membrane which resulted in efficient rehydration by the viscous sample in lesser amount of time. Hence, the first cassette 802 was found to be more effective compared to the second cassette 804 in handling viscous samples.

This example demonstrated that both the first cassette 802 and the second cassette 804 can be used for the effective stabilization of clinical samples with viscosity ranging from 1 cP to 4 cP. It was also observed that both the first cassette 802 and the second cassette 804 can handle large volume of the clinical samples. Moreover, the time frames suggested that the time required for the stabilization of the viscous sample was relatively lesser for the first cassette 802 and the second cassette 804 compared to the corresponding control devices 801. Hence, the first cassette 802 and the second cassette 804 can be looked upon as an efficient way of collecting clinical samples. Overall, the first cassette 802 design displayed slightly better stabilization chemistry compared to the second cassette 804 design.

### Example 3

### Effect of Different Materials of Fabrication

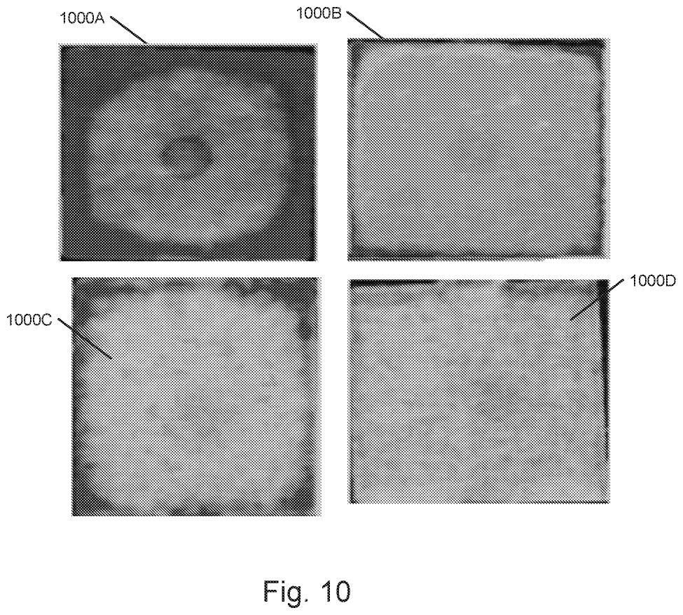

In this example, effect of different materials, in particular, storage membranes and distributor layer were studied. Four devices were constructed—a first control device comprising the top layer coupled to a standard 17 membrane acting as the storage membrane; a second control device comprising the top layer coupled to a nitrocellulose membrane acting as the storage membrane; a third cassette comprising the top layer, a standard 17 membrane acting as both the distributor layer and the storage membrane; and a fourth cassette comprising the top-layer, a standard 17 membrane acting as the distributor layer and a nitrocellulose membrane acting as the storage membrane. The fluid injected in the top layer of each of the first control device, the second control device, the third cassette, and the fourth cassette were deionized water with viscosity of 1 cP. Results of the study was as illustrated in FIG. 10.

In the first control device, indicated by illustration 1000A, the fluid rapidly took out the dye on the standard 17 membrane towards the edges. This confirmed that the incoming fluid and the dye (substitute for reagents) mostly interact near boundaries of the standard 17 membrane.

In the second control device, indicated by illustration 1000B, the fluid rehydrated the nitrocellulose membrane at a relatively slower pace and almost all the dye was pushed towards the boundaries. The slower pace was attributed to lesser fluidic capacity of the nitrocellulose membrane compared to the standard 17 membrane.

In the third cassette, indicated by illustration 1000C, as the distributor layer and the storage membrane were both the same, the results were found to be similar to illustration 1000A, i.e., the dye was pushed towards the edges as indicated by the darker regions. However, in the fourth cassette, indicated by illustration 1000D, as the distributor layer and the storage membrane were fabricated from different material having different flow rates for the fluid (substitute for clinical sample), it demonstrated a uniform rehydration of the nitrocellulose membrane. In addition, concentrated boundaries which indicate sample-reagent interaction were absent in the fourth cassette. Thus, the stabilization chemistry was better and improved compared to other cases.

Therefore, the clinical sample storage cassette of the present subject matter can be used for storage of clinical samples of quantities greater than 1 mL with higher stability and uniformity. Further, it can also be used for stabilization and storage of any kind of clinical sample. The clinical sample storage cassette allows for uniform distribution of the clinical sample over the storage membrane, thus providing improved stabilization and shelf-life of the clinical sample.

Although the subject matter has been described in considerable detail with reference to certain examples and implementations thereof, other implementations are possible. As such, the scope of the present subject matter should not be limited to the description of the preferred examples and implementations contained therein.

1. A clinical sample storage cassette comprising:
a top layer comprising a first side to receive a clinical sample and a second side opposite to the first side to couple with a distributor layer;
the distributor layer coupled to the top layer to receive the clinical sample from the top layer, wherein the distributor layer comprises a first distributor side coupled to the second side of the top layer and a second distributor side coupled to a storage membrane to transfer the clinical sample from top layer to the storage membrane; and
the storage membrane to receive the clinical sample from the distributor layer and to store the clinical sample, wherein a flow rate of the clinical sample through the distributor layer is greater than a flow rate of the clinical sample through the storage membrane to allow for uniform distribution and storage of the clinical sample in the storage membrane.

2. The clinical sample storage cassette as claimed in claim 1, wherein the top layer comprises a sample inlet port for introduction of the clinical sample into the clinical sample storage cassette and wherein the sample inlet port extends from the first side to the second side of the top layer.

3. The clinical sample storage cassette as claimed in claim 1, wherein the distributor layer comprises a plurality of sub-distributor layers, wherein the plurality of sub-distributor layers comprises:
a top-most sub-distributor layer having the first distributor side coupled to the second side of the top layer;
a bottom-most sub-distributor layer having the second distributor side coupled to the storage membrane; and
a plurality of intermediate layers disposed between the top-most sub-distributor layer and the bottom-most sub-distributor layer.

4. The clinical sample storage cassette as claimed in claim 3, wherein each of the plurality of sub-distributor layers comprises distributor ports.

5. The clinical sample storage cassette as claimed in claim 4, wherein a number of distributor ports from the top-most sub-distributor layer, through each of the plurality of intermediate layers, and to the bottom-most sub-distributor layer increases exponentially by an even power of 2.

6. The clinical sample storage cassette as claimed in claim 4, wherein the top-most sub-distributor layer comprises 2 distributor ports and the bottom-most sub-distributor layer comprises 16 distributor ports.

7. The clinical sample storage cassette as claimed in claim 1, wherein the distributor layer is porous.

8. The clinical sample storage cassette as claimed in claim 1, wherein the top layer is fabricated from acrylic plastic.

9. The clinical sample storage cassette as claimed in claim 1, wherein the distributor layer is fabricated from one of glass fibers and acrylic plastic.

10. The clinical sample storage cassette as claimed in claim 1, wherein the top layer, the distributor layer, and the storage membrane are coupled to each other by a Pressure Sensitive Adhesive (PSA) tape.

11. The clinical sample storage cassette as claimed in claim 1, wherein the distributor layer comprises a fluid channel, wherein the fluid channel comprises:
a main channel to receive the clinical sample from the top layer; and
a plurality of sub-channels extending away from the main channel to receive the clinical sample from the main channel.

12. An assembly for storage of clinical sample comprising:
a clinical sample storage cassette comprising:
a top layer comprising a first side to receive a clinical sample and a second side opposite to the first side to couple with a distributor layer;
the distributor layer coupled to the top layer to receive the clinical sample from the top layer, wherein the distributor layer comprises a first distributor side coupled to the second side of the top layer and a second distributor side coupled to a storage membrane to transfer the clinical sample from top layer to the storage membrane; and
the storage membrane to receive the clinical sample from the distributor layer and to store the clinical sample, wherein a flow rate of the clinical sample through the distributor layer is greater than a flow rate of the clinical sample through the storage membrane to allow for uniform distribution and storage of the clinical sample in the storage membrane; and
a container to hold the clinical sample storage cassette; and
a desiccant pack provided within the container to dry the storage membrane.

13. The assembly as claimed in claim 12, wherein the distributor layer comprises a plurality of sub-distributor layers, wherein the plurality of sub-distributor layers comprises:
a top-most sub-distributor layer having the first distributor side coupled to the second side of the top layer;
a bottom-most sub-distributor layer having the second distributor side coupled to the storage membrane; and
a plurality of intermediate layers disposed between the top-most sub-distributor layer and the bottom most sub-distributor layer.

14. The assembly as claimed in claim 12, wherein the distributor layer comprises a fluid channel, wherein the fluid channel comprises:
a main channel to receive the clinical sample from the top layer; and
a plurality of sub-channels extending away from the main to receive the clinical sample from the main channel.

15. The assembly as claimed in claim 12, wherein the container is a centrifuge tube.

16. A method for collection and analysis of clinical sample comprising:
receiving by a clinical sample storage cassette the clinical sample, wherein the clinical sample storage cassette is held in a container, wherein the clinical sample storage cassette comprises:
a top layer comprising a first side to receive the clinical sample and a second side opposite to the first side to couple with a distributor layer;
the distributor layer coupled to the top layer to receive the clinical sample from the top layer, wherein the distributor layer comprises a first distributor side coupled to the second side of the top layer and a second distributor side coupled to a storage membrane to transfer the clinical sample from top layer to the storage membrane; and
the storage membrane to receive the clinical sample from the distributor layer and to store the clinical sample, wherein a flow rate of the clinical sample through the distributor layer is greater than a flow rate of the clinical sample through the storage membrane to allow for uniform distribution and storage of the clinical sample in the storage membrane;
drying the storage membrane by a desiccant pack provided within the container;
receiving by the container an elution buffer;
centrifuging the container to extract the clinical sample from the storage membrane to obtain a remnant fluid comprising a mixture of the elution buffer and the clinical sample; and
providing the remnant fluid for analysis.
